(19) United States
(12) Patent Application Publication
(10) Pub. No.: US 2023/0390462 A1
(43) Pub. Date: Dec. 7, 2023

(54) OSTEON TEMPLATES FOR BONE TISSUE ENGINEERING

(71) Applicant: Vestlandets Innovasjonsselskap AS, Bergen (NO)

(72) Inventors: Ahmad Rashad Saad Mohamed Elsebahy, Bergen (NO); Kamal Babikeir Eln Mustafa, Bergen (NO)

(21) Appl. No.: 18/032,307

(22) PCT Filed: Oct. 18, 2021
(86) PCT No.: PCT/EP2021/078856

§ 371 Date: Apr. 17, 2023

(30) Foreign Application Priority Data
Oct. 16, 2020 (GB) 2016465.3

(51) Int. Cl. A61L 27/54; A61L 27/52; A61L 27/36; A61L 27/38; A61F 2/30; B33Y 80/00; B33Y 10/00

(52) U.S. Cl. CPC A61L 27/54; A61L 27/52; A61L 27/365; A61L 27/3808; A61F 2/30965; A61F 2/30942; B33Y 80/00; B33Y 10/00; A61L 2300/412; A61F 2002/30118; A61F 2002/30116; A61F 2002/2817

(57) ABSTRACT

There is provided an osteon template for tissue engineering, including a first plurality of vasculogenic filaments including a first hydrogel and vessel-forming cells, and a second plurality of osteogenic filaments including a second hydrogel and bone-forming cells, wherein the first plurality of vasculogenic filaments is arranged alternately with the second plurality of osteogenic filaments in a concentric arrangement.

The present invention relates to devices and methods for tissue engineering using an osteon template.

Tissue engineering generally relates to the growth of new tissues, or organs, from living cells using an engineered structure such as a template or a scaffold as a support during cellular growth. Tissue engineering techniques may be used to produce an organ or tissue graft for implantation back into a donor host. Tissue engineering frequently involves stem cells; implanting stem cells in an appropriate location can generate bone, tendon and cartilage. Applications include dermal wound healing and repair of cartilage, ligament or bone in vivo as well as the study of such processes in vitro.

Templates containing biocompatible materials seeded with stem cells can be implanted into the body in order to aid bone regeneration or repair. However the failure rate of bone grafts can be high. In bone tissue engineering, the osteogenic and angiogenic potential of stem cells in 3D structural systems has been demonstrated in vitro and in vivo. A classic tissue engineering treatment uses bone cells carried by a synthesized template to accelerate healing procedures. The environment for bone regeneration is very complicated, including different cell types, growth factors, nutrition supply and mechanical stimulation.

It has been found that the ability of nutrients to perfuse through the tissue engineering template and for waste products to be removed from the template aids in the ability of the bone to develop in the template region. This is particularly important when providing a template to aid in the development of compact cortical bone due to the containment within the bone material of osteocyte cells in units called osteons. The osteocyte cells are instrumental in bone fracture healing and depend on receiving nutrients and dispelling waste in order to carry out the process of bone healing.

It is known to form bone tissue engineering templates from hydrogels comprising endothelial cells, such as human umbilical vein endothelial cells (HUVEC), and stem cells, such as human mesenchymal stem cells (hMSC). It has recently been proposed to mimic the spatial pattern of HUVECs and hMSCs found in native osteons based on the use of extrusion-based 3D bioprinting. Researchers in this field have attempted to print HUVEC- and hMSC-laden fibrin hydrogels as “bio-inks” in an osteon-like construct. However it remains to be proven whether such constructs provide sufficient neovascularisation to successfully achieve bone tissue fabrication.

There remains a need for a bone tissue engineering template that enables vascularisation and reliable bone growth.

According to a first aspect of the present invention, there is provided an osteon template for tissue engineering, comprising:
a first plurality of vasculogenic filaments comprising a first hydrogel and vessel-forming cells and a second plurality of osteogenic filaments comprising a second hydrogel and bone-forming cells;
wherein the first plurality of vasculogenic filaments is arranged alternately with the second plurality of osteogenic filaments in a concentric arrangement.

In tissue engineering it is common to maximise the amount of osteogenic material in order to stimulate bone growth. However the inventors have appreciated that, in native bone tissue, most cells are within a limited distance from blood capillaries to ensure sufficient diffusion of oxygen, perfusion of nutrients, and removal of waste products. By arranging the vasculogenic and osteogenic filaments alternately, it can be ensured that perfusion processes are promoted whilst also providing sufficient bone-forming constituents to support efficient bone growth. The disclosed concentric arrangement therefore achieves a balance between osteogenic potential and vascularisation.

From the above discussion it will be understood that each one (or several) of the first plurality of vasculogenic filaments is arranged alternately with another one (or several) of the second plurality of osteogenic filaments, meaning an alternation from one type of filament to the other and back again. Of course the alternation may be repeated, in a regular or irregular pattern. This alternation ensures that the osteogenic filaments in the concentric arrangement benefit from the perfusion processes provided by the vasculogenic filaments whilst also providing sufficient density and distribution of bone-forming cells to support efficient bone growth. Thus, it will be understood that the concentric arrangement comprises: one or more of the first plurality of vasculogenic filaments followed by one or more of the second plurality of osteogenic filaments and then another one or more of the first plurality of vasculogenic filaments; or one or more of the second plurality of osteogenic filaments followed by one or more of the first plurality of vasculogenic filaments and then another one or more of the second plurality of osteogenic filaments.

It is expected that bone growth within the template will be improved by increasing the blood perfusion and vascularisation throughout the template. The inventors have further recognised several ways to enhance the perfusability of such an osteon template.

In at least some embodiments, the template further comprises at least one radial channel emanating from the centre of the concentric arrangement to interrupt the first and second pluralities of vasculogenic and osteogenic filaments. The at least one radial channel provides an in-plane pathway for materials to be transported through the concentric arrangement, allowing for better diffusion in vitro and for tissue integration and vascularisation in vivo. The at least one radial channel defines a gap between adjacent vasculogenic filaments and between adjacent osteogenic filaments (i.e. in the circumferential direction). The gap may be much larger in size than the diameter or width of the filaments, for example an order of magnitude larger. For example, the vasculogenic and osteogenic filaments may have a diameter of the order of 200 μm and the gap may be about 2 mm wide.

In at least some embodiments, the template comprises a number n (where n>1) of radial channels interrupting the first and second pluralities of vasculogenic and osteogenic filaments so as to divide each of the vasculogenic and osteogenic filaments into n arcuate segments. The radial channels therefore define a gap between adjacent arcuate segments to allow for perfusion. As mentioned above, the gap could be about 1-2 mm wide.

In at least some embodiments, the concentric arrangement may include the first and second plurality of filaments arranged concentrically in multiple incomplete arcs of a circle, interrupted by the radial channels, such that multiple arcuate segments of a circle are formed, e.g. each having coinciding centres of curvature. The centre of curvature of the arcuate segments may coincide with the centre of the concentric arrangement. The radial channels may extend from the centre to the outer periphery of the concentric arrangement.

The greater the number of radial channels, the more pathways that are available for perfusion, but an excessive number may start to detrimentally reduce the amount of osteogenic material available in the template. In some embodiments the number n is chosen to be in the range of 2-10, for example a template comprising 2, 4, 6 or 8 radial channels. In some embodiments the number n is chosen depending on the diameter of the template. This means that more radial channels may be included for larger templates.

In at least some embodiments, in addition or alternatively, the template further comprises a central void generally at the centre of the concentric arrangement. The presence of a central void allows for out-of-plane transportation of materials to/from the centre of the concentric arrangement, again allowing for better diffusion in vitro and for tissue integration and vascularisation in vivo. It will be appreciated that the central void may not be exactly centred in relation to the concentric arrangement and the template may be not be rotationally symmetrical. In combination with any embodiment wherein the template includes one or more radial channels, the radial channel(s) may emanate from the central void and the void conveniently allows for the radial pathways to meet and for materials to be exchanged between them. The central void may be larger in size than the diameter or width of the filaments, for example an order of magnitude larger. For example, the vasculogenic and osteogenic filaments may have a diameter of the order of 200 μm and the central void may have a diameter of 3-4 mm.

The vasculogenic and/or osteogenic filaments may be formed as homogeneous filaments. In at least some embodiments, the osteogenic filaments have a solid structure formed by the second hydrogel so as to maximise the distribution of bone-forming cells. The vasculogenic filaments may also have a solid structure formed by the first hydrogel. However, the inventors have recognised that it can be beneficial for the vasculogenic filaments to be formed as heterogeneous filaments. In at least some embodiments, in addition or alternatively, one or more of the first plurality of vasculogenic filaments consist of a hollow core surrounded by a shell comprising the first hydrogel and vessel-forming cells. Such a core-shell structure is expected to assist with perfusion as materials can be transported circumferentially through the concentric arrangement via the hollow cores of the vasculogenic filaments. In combination with any embodiment wherein the template includes one or more radial channels, the number of radial channels may be reduced in view of the perfusion provided by the hollow cores. This can help to maximise the volume of osteogenic filaments in the template whilst maintaining its perfusive nature. The use of a core-shell structure for the vasculogenic filaments provides a more dynamic environment for the template which may assist with circulation e.g. during drug testing.

The inventors have further recognised that the hollow core of the vasculogenic filaments may be exploited for other purposes. For example, the hollow core may enable drug delivery and assist with drug testing. In at least some of these embodiments, one or more bioreactor tubes are arranged to extend through the hollow core of at least some of the vasculogenic filaments. The bioreactor tubes can therefore emulate the circulation of blood around the osteon template, which may be useful for drug testing, for example.

The alternating vasculogenic and osteogenic filaments may be arranged with an osteogenic filament at the radial interior of the concentric arrangement and/or an osteogenic filament at the radial exterior of the concentric arrangement. This may be expected to maximise the number of bone-forming cells present in the template for bone growth. However, the inventors have recognised that it is beneficial for the osteogenic filaments to be sandwiched between vasculogenic filaments so as to promote vascularisation. Thus in at least some embodiments, in addition or alternatively, one or more (and preferably each) of the osteogenic filaments in the concentric arrangement is bounded by a vasculogenic filament disposed on a radially inward side and another vasculogenic filament disposed on a radially outward side.

While it is an aim of the embodiments disclosed herein to promote vascularisation, successful tissue engineering also relies on a sufficient number of bone-forming cells being present. The vasculogenic and osteogenic filaments may be formed to have the same size and diameter. But, to maximise bone growth potential, it is preferable that the osteogenic filaments are larger than the vasculogenic filaments. Thus, in at least some embodiments, the vasculogenic filaments have a first diameter and the osteogenic filaments have a second diameter that is greater than the first diameter. The extent to which the osteogenic filaments are larger than the vasculogenic filaments can be a balance between seeding bone growth and promoting vascularisation. The inventors have appreciated that, in native bone tissue, most cells are within a maximum distance of 100-200 μm from blood capillaries. The second diameter may therefore be chosen to be in the range of 200-400 microns. In at least some embodiments, the first diameter is in the range of 50-200 microns and the second diameter is in the range of 200-400 microns.

In various embodiments the first hydrogel may be substantially the same as the second hydrogel. Any suitably biocompatible hydrogel material may be used. Examples of synthetic materials capable of forming hydrogels suitable for tissue engineering include poly(ethylene oxide), poly(vinyl alcohol), poly(acrylic acid), poly(propylene fumarate-co-ethylene glycol), and polypeptides. Examples of natural hydrogels include agarose, alginate, chitosan, collagen, fibrin, gelatin, and hyaluronic acid. The molecular weight, material concentration, choice of cross-linker and gelling conditions may all be taken into consideration to achieve a desired stiffness and/or stability for the first and second hydrogel. Furthermore, at least in embodiments wherein the vasculogenic and osteogenic filaments are deposited by 3D printing techniques, the viscosity of the hydrogel may be taken into consideration.

The first and/or second hydrogel may comprise one or more additives. For example, the first hydrogel may include nanocellulose to guide alignment of the vessel-forming cells and angiogenesis tube formation. For example, the second hydrogel may include needle-like hydroxyapatite nanoparticles to guide osteogenic differentiation of the bone-forming cells. Furthermore, or in the alternative, an additive may be chosen to alter the stiffness of the first and/or second hydrogel. More generally, the first hydrogel may be chosen or engineered as a “soft” hydrogel and the second hydrogel may be chosen or engineered as a “stiff” or “rigid” hydrogel. Thus, in at least some embodiments, the vasculogenic filaments comprise the first hydrogel having a first stiffness and the osteogenic filaments comprise the second hydrogel having a second stiffness that is greater than the first stiffness. For example, the second hydrogel may include high aspect ratio (e.g. needle-like) hydroxyapatite nanoparticles, which may optionally be magnesium-doped.

The vasculogenic and osteogenic filaments may be stiff enough that the concentric arrangement is self-supporting. However, in various embodiments the filaments are interrupted by one or more radial channels and this may make it more difficult for the concentric arrangement to hold its shape. Especially in those embodiments wherein multiple radial channels split the filaments into separate arcuate segments, a base layer may be needed to support the arcuate segments in position relative to one another, e.g. so that the radial channels are maintained open. In at least some embodiments, the template further comprises a base layer and the concentric arrangement is disposed on the base layer. The base layer may be formed from a hydrogel, which could optionally match the first and/or second hydrogel. As before, the hydrogel may include one or more additives to adjust its material properties.

The osteon template disclosed herein may find use as a substantially planar template, for example in drug testing and basic cell research. Furthermore, the osteon template may be formed as a three-dimensional template by depositing the filaments as high aspect ratio sheets. However, it is typical in additive manufacturing processes such as 3D printing for the filaments to be deposited with a substantially unitary aspect ratio, for example filaments having a generally circular cross-section as a result of extrusion at the point of deposition. An advantage of additive manufacturing processes is that multiple layers can be deposited in quick succession to build up a three-dimensional structure. In at least some embodiments, the osteon template is a three-dimensional structure comprising multiple layers. In one or more embodiments, the concentric arrangement forms a first filament layer, the osteon template comprising one or more further filament layers formed from such concentric arrangements and stacked on the first filament layer to form a three-dimensional osteon template comprising a plurality of the filament layers. A concentric arrangement may have a polygonal geometry but a circular geometry is preferred to mimic the shape of a natural osteon. In at least some embodiments the three-dimensional osteon template is generally cylindrical.

In at least some of these embodiments, one or more radial channels are formed in each filament layer and the one or more radial channels are aligned in each of the plurality of filament layers to define an axially and radially extending channel in the three-dimensional osteon template.

In at least some of these embodiments, in addition or alternatively, each filament layer comprises a central void and the filament layers are stacked such that the central voids define an axial channel at the centre of the three-dimensional osteon template.

In at least some of these embodiments, in addition or alternatively, an additional support layer (e.g. a support layer of hydrogel) is arranged amongst the plurality of the filament layers. For example, an additional support layer is arranged above the first filament layer, or above one or more of the further filament layers, and at least one further filament layer is arranged on top of the additional support layer. The additional support layer may be formed from a hydrogel, and the hydrogel can optionally match the first and/or second hydrogel. As before, the hydrogel may include one or more additives to adjust its material properties.

It will be appreciated that the three-dimensional osteon template disclosed above may be engineered to substantially match a natural osteon. For example, the osteon template may be made with a diameter that substantially matches the diameter of natural osteons in a target bone to be grown using the osteon template. In some further embodiments of the present invention, several osteon templates may be arranged in an array to mimic the natural structure of cortical bone. For example, multiple osteon templates as disclosed herein may be arranged in a generally circular and/or concentric array to form an osteogenic model. Such an osteogenic model may therefore mimic the natural complexity of bone structure. The osteogenic model may be self-supporting in at least some examples.

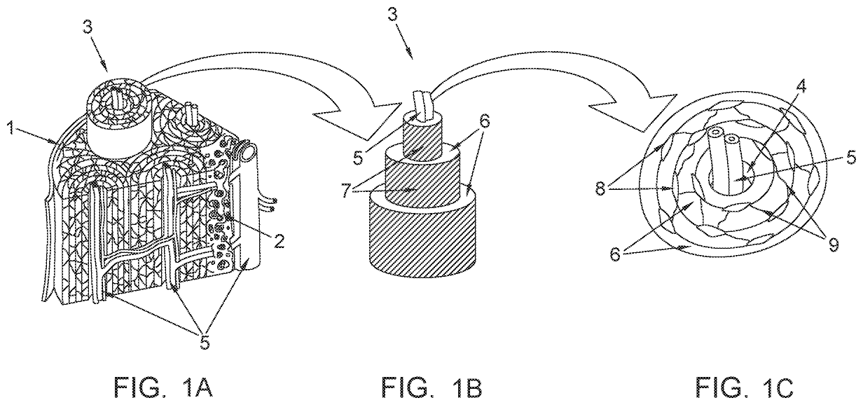

It has previously been proposed by the inventors to use a three-dimensional scaffold as a physical support for in vivo growth of stem cells and/or vessel-forming cells. The geometry of the scaffold may be designed to match a natural bone environment, as is described in WO2018/162764. The inventors have now recognised that the osteon templates disclosed herein may take advantage of such a three-dimensional scaffold, or any other suitable tissue engineering scaffold, to support a plurality of the osteon templates in a desired arrangement to form an osteogenic model. The arrangement of the osteon templates in the osteogenic model may depend on its application, for example a simply linear or circular array may be sufficient for a three-dimensional model to be used for in vitro drug testing. At least in examples where the three-dimensional model is to be used to mimic the anatomical structure of natural bone, e.g. for in vivo bone growth, the osteon templates may be arranged in a concentric array i.e. in a similar arrangement to natural osteons in compact bone. This is described further below with reference to FIG. 1.

According to a further aspect of the present invention, there is provided an osteogenic model comprising a three-dimensional scaffold and a plurality of the osteon templates as disclosed herein arranged to be supported by the scaffold in a generally concentric array. It will be understood that a concentric array means multiple “rings” of osteon templates surrounding a common centre point, albeit that the “rings” may not be circular. The scaffold therefore assists with supporting the osteon templates in an array to mimic cortical bone. A suitable three-dimensional scaffold is described in WO2018/162764, the contents of which are hereby incorporated by reference.

In at least some embodiments, the three-dimensional scaffold comprises a first set of one or more walls and a second set of one or more walls arranged to substantially surround the first set of one or more walls with a spacing between the first and second sets of one or more walls defining a cavity between the walls, and wherein the concentric array is arranged in the cavity between the walls. This means that the cavity can be suitably dimensioned to control the concentric array of osteon templates, for example with dimensions designed to match natural bone in a target.

In at least some embodiments, the three-dimensional scaffold comprises: an inner portion comprising a channel surrounded by a first set of one or more walls; an outer portion comprising a second set of one or more walls and arranged such that the second set of one or more walls substantially surrounds the first set of one or more walls with a spacing between the first and second sets of one or more walls defining a cavity between the inner portion and the outer portion. Optionally the scaffold includes a base portion connecting the inner portion and the outer portion. In these embodiments, the concentric array of osteon templates may be arranged in the cavity between the inner portion and the outer portion. This means that the concentric array can be controlled to match the compact bone in natural bone, which does not extend to the centre of the bone. As is described in WO2018/162764, the inner channel can be dimensioned to match the medullary cavity within the cancellous (spongy bone) that is surrounded by compact bone in natural bone. It is the medullary cavity that enables blood vessels to carry a flow of oxygen through the centre of the bone and take away waste products. A three-dimensional scaffold of this type may assist with vascularisation of the osteon templates.

In any of the embodiments relating to an osteogenic model comprising a three-dimensional scaffold, the scaffold may be made from any suitable (e.g. biocompatible) material having sufficient strength to support the osteon templates. Suitable material may include ceramics, polymers, metals and even hydrogels. However, the scaffold is preferably stronger and/or stiffer than the osteon templates. It is preferable that the scaffold is formed from a polymeric material (including polymer-based composites), whether natural or synthetic. The scaffold may be formed from one or more polymeric materials including, but not limited to: polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, chitin, chitosan, poly(L-lactic acid), poly(lactide-co-glycolide), poly(hydroxybutyrate-co-valerate), and copolymers, terpolymers, or combinations or mixtures of the above polymeric materials.

In some embodiments, the walls of the scaffold may be substantially solid. In other embodiments, the walls of the scaffold may include apertures or may be made as a micro-filament mesh.

Also as described in WO2018/162764, the scaffold may conveniently be made using additive manufacturing techniques. In at least some embodiments the scaffold is made using a 3D fiber deposition (3DF) method. In at least some embodiments the scaffold is made using a computer-controlled fabrication technique, such as a rapid prototyping (RP) method. Suitable RP methods include 3D printing (e.g. fused deposition modelling), selective laser sintering, and other layer-by-layer techniques. Using such techniques the scaffold design can be scaled up or down to any desired dimensions (albeit within the limit of the RP machine's resolution). In at least some embodiments the scaffold is made from/to a customised reproducible design recorded in a computer-aided design (CAD) model. This means that bespoke scaffolds can be produced quickly and cost effectively, to create an osteogenic model that mimics a target bone environment.

Regardless of whether a three-dimensional scaffold is employed to support the osteon templates in an osteogenic model, the osteon templates themselves may be produced rapidly and precisely by additive manufacturing techniques such as 3D printing.

According to a further aspect of the present invention, there is provided a method of making the osteon template of any of the embodiments disclosed herein, using additive manufacturing to deposit at least the first plurality of vasculogenic filaments and the second plurality of osteogenic filaments.

According to a further aspect of the present invention, there is provided a method of manufacturing an osteon template, the method comprising:
using an additive manufacturing process to deposit a concentric arrangement comprising a first plurality of vasculogenic filaments and a second plurality of osteogenic filaments, the first plurality of vasculogenic filaments being arranged alternate with the second plurality of osteogenic filaments in the concentric arrangement;
wherein the first plurality of vasculogenic filaments comprises a first hydrogel and vessel-forming cells and the second plurality of osteogenic filaments comprises a second hydrogel and bone-forming cells.

As described above, it will be understood that each one (or several) of the first plurality of vasculogenic filaments is arranged alternately with another one (or several) of the second plurality of osteogenic filaments, meaning an alternation from one type of filament to the other and back again. Of course the alternation may be repeated, in a regular or irregular pattern. Thus, it will be understood that the method may comprise: using an additive manufacturing process to deposit the concentric arrangement so as to comprise: one or more of the first plurality of vasculogenic filaments followed by one or more of the second plurality of osteogenic filaments and then another one or more of the first plurality of vasculogenic filaments; or one or more of the second plurality of osteogenic filaments followed by one or more of the first plurality of vasculogenic filaments and then another one or more of the second plurality of osteogenic filaments.

It will be appreciated that additive manufacturing techniques allow for rapid and customisable production of osteon templates. Suitable additive manufacturing processes may include 3D printing, preferably microfluidic 3D bioprinting.

The additive manufacturing process may involve the use of an extrusion-based 3D printer comprising several dispense heads. This allows for the printing of different materials for creation of the osteon template using a single 3D printer. For example, by loading a bone-forming hydrogel in a first cartridge of the 3D printer associated with a first dispense head, a vascular-forming hydrogel in a second cartridge of the 3D printer associated with a second dispense head, and a structural bioink [such as a biocompatible thermoplastic, e.g. polycaprolactone (PCL), or a cell-free hydrogel] in a third cartridge of the 3D printer associated with a third dispense head, the printer can dispense layers of the three hydrogels/bioinks according to a CAD design.

In some embodiments, using an additive manufacturing process to deposit the concentric arrangement comprises: using a 3D printing process in which the first plurality of vasculogenic filaments is deposited by a first dispense head and the second plurality of osteogenic filaments is deposited by a second dispense head. For example, the method may comprise controlling the first and second dispense heads to dispense the vasculogenic and osteogenic filaments alternately to achieve the concentric arrangement. In at least some examples, the 3D printing process comprises using a further dispense head to deposit a base layer, such as a base layer of a cell-free structural material. Examples of a suitable structural material include biocompatible thermoplastics, e.g. polycaprolactone (PCL), or hydrogel. The method may comprise depositing the concentric arrangement on top of the base layer.

The additive manufacturing process, e.g. 3D printing, may be controlled (e.g. by controlling different dispense heads as described above) to create a concentric arrangement with enhanced perfusion features as already described above. In some embodiments, the method further comprises: controlling the additive manufacturing process to form at least one radial channel emanating from the centre of the concentric arrangement to interrupt the first and second pluralities of vasculogenic and osteogenic filaments.

In some embodiments, in addition or alternatively, the method further comprises: controlling the additive manufacturing process to form one or more of the first plurality of vasculogenic filaments as a core-shell structure consisting of a hollow core surrounded by a shell comprising the first hydrogel and vessel-forming cells. In at least some embodiments, the method comprises: controlling a coaxial dispense head to dispense one or more of the first plurality of vasculogenic filaments as a core-shell structure. In such embodiments, the method may further comprise: controlling the additive manufacturing process to form a number n of radial channels, wherein n is determined based on how many of the vasculogenic filaments are formed with the core-shell structure. This means that the osteon template can be tailored for perfusion.

In some embodiments, in addition or alternatively, the method further comprises: determining a diameter for the osteon template based on a target bone to be grown using the osteon template; and controlling the additive manufacturing process such that the concentric arrangement is formed with said diameter. This means that the osteon template can be tailored dependent on its intended application. In such embodiments, the method may further comprise: controlling the additive manufacturing process to form a number n of radial channels, wherein n is determined based on the diameter of the concentric arrangement. This means that the perfusive nature of the osteon template can be tailored based on its size.

In any of the embodiments described above, the vessel-forming cells in the vasculogenic filaments may be selected as human or animal cells. Suitable vessel-forming cells may include stem cells that can be differentiated to endothelial cells, endothelial cells (e.g. alone or with fibroblasts), or neural progenitor cells. In some preferred embodiments the vessel-forming cells comprise endothelial cells, e.g. with or without growth factors (e.g. VEGF). For example, the vessel-forming cells may consist of endothelial cells such as human umbilical vein endothelial cells (HUVEC).

In any of the embodiments described above, the bone-forming cells in the osteogenic filaments may be selected as human or animal cells. Suitable bone-forming cells may include pluripotent stem cells, mesenchymal stem cells (also known as mesenchymal stromal cells), osteoprogenitor cells, osteoblasts, and osteocytes. In some preferred embodiments the bone-forming cells comprise mesenchymal stem cells. For example, the bone-forming cells may consist of mesenchymal stem cells such as human bone marrow mesenchymal stem cells (hBMSC).

Some embodiments of the present invention will now be described, by way of example only, and with reference to the accompanying drawings, in which:

FIG. 1 shows a schematic of the native osteon structure within compact bone.

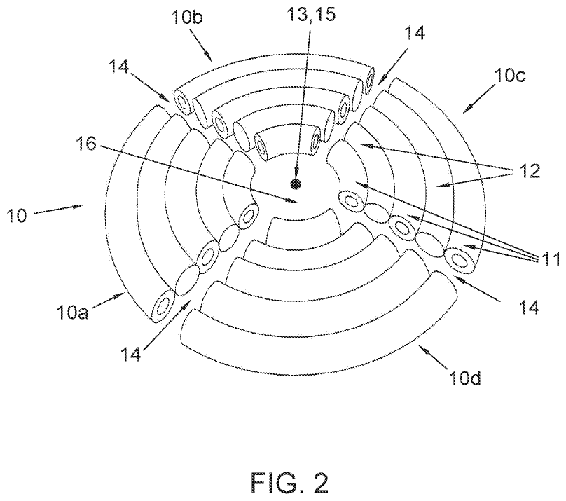

FIG. 2 schematically shows an embodiment of an osteon template for tissue engineering comprising a concentric arrangement.

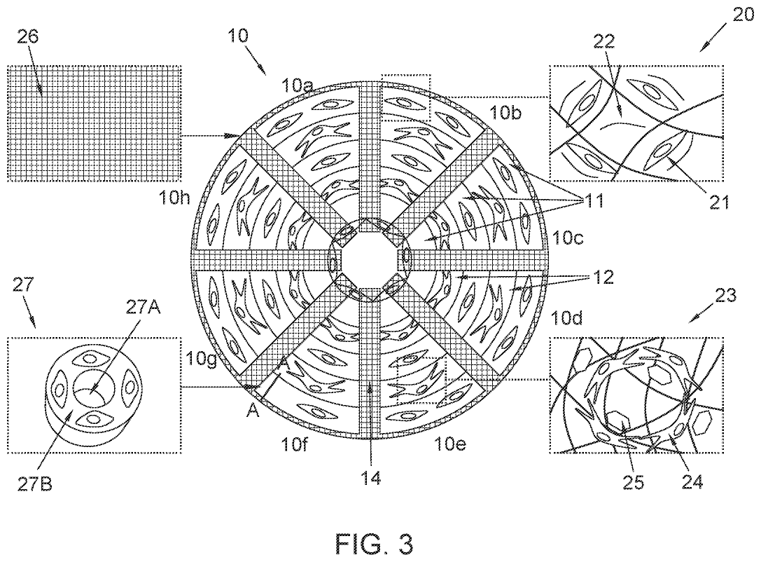

FIG. 3 shows a schematic of the constituents of the osteon template according to another embodiment.

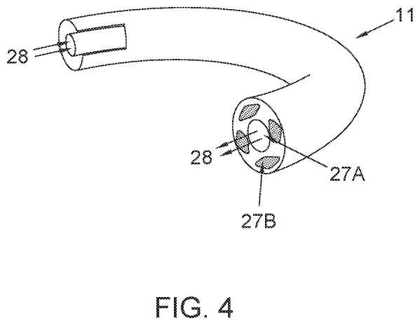

FIG. 4 schematically shows a vascular-forming hydrogel filament of the osteon template.

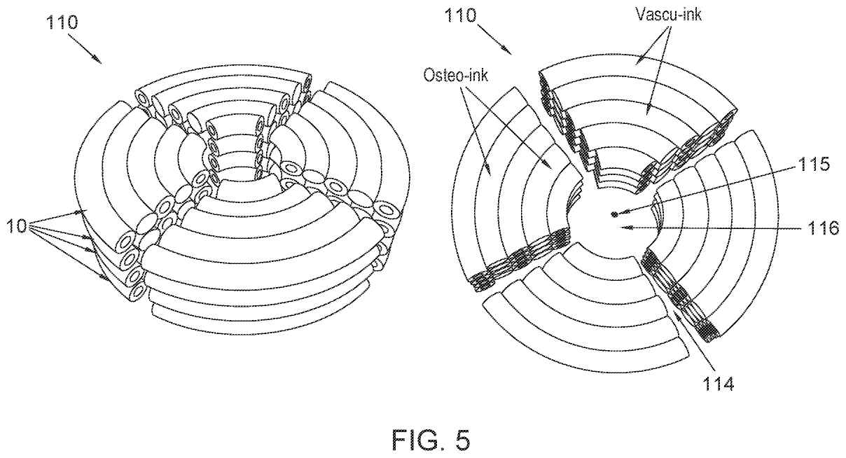

FIG. 5 schematically shows an osteon template for tissue engineering comprising multiple concentric arrangements.

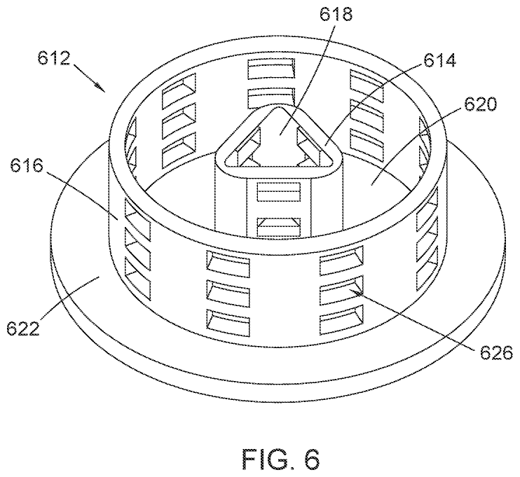

FIG. 6 shows a polymer bone graft template in which osteon templates may be disposed.

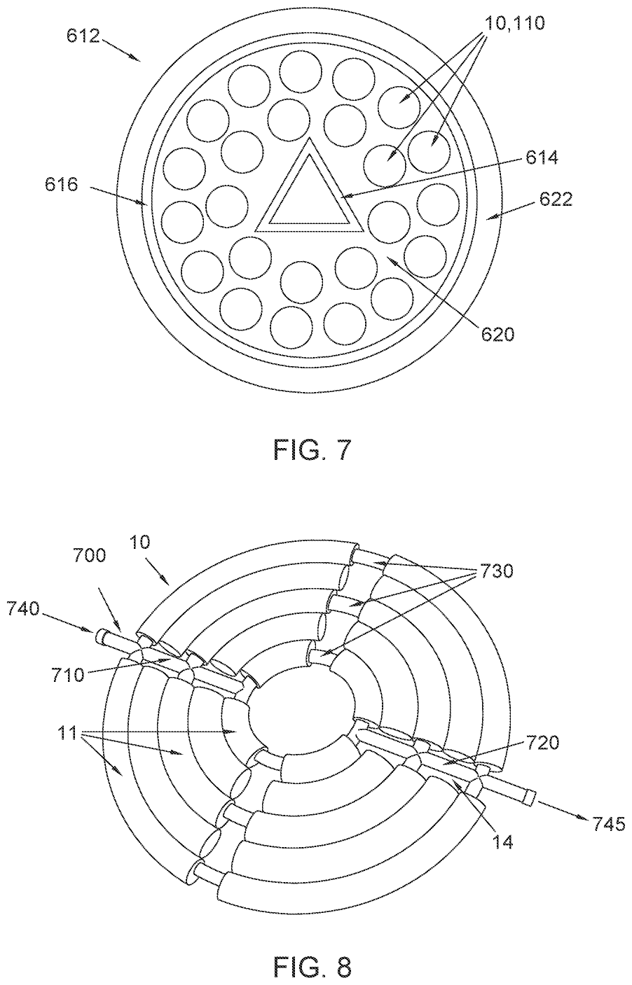

FIG. 7 shows a polymer bone graft with a plurality of osteon templates disposed within.

FIG. 8 schematically shows an example of an osteon template used with a bioreactor.

FIG. 1 shows the hierarchical structure of compact bone. The bone comprises compact cortical bone 1, surrounding spongy cancellous bone 2 with a central channel accommodating blood vessels 5. The cortical bone 1 consists of repeated functional units called osteons 3. The osteons 3 are cylindrical structures comprising a central osteonic canal (Haversian canal) 4, containing blood vessels 5, surrounded by lamellae 6 of a compact matrix of collagen 7 and bone mineral. As seen in FIG. 1a, the osteons 3 within cortical bone 1 structure are typically aligned parallel to the long axis of the bone so that they are aligned in the direction of applied stress. The osteons 3 therefore provide strength to the bone and aid the bone in resisting bending and fracture. FIG. 1b shows an exploded diagram of the lamellae structure of the osteon 3. Each lamella 6 contains collagen fibres aligned within one lamella 6, but aligned orthogonally to the collagen fibres of the neighbouring lamella 6 for increased strength. As seen in FIG. 1c, many osteocytes 8 are found within the lamellae 6 of each osteon 3. Osteocytes are cells derived from osteoblasts whose function is to control and carry out bone regeneration, through bone resorption and deposition, in order to maintain healthy bone that meets the demands placed on it by its position within the body. As seen in FIG. 1c, the osteocytes 8 within a single osteon 3 are connected to each other via canaliculi 9, capillaries for transporting blood. Nutrients and waste products are exchanged via the canaliculi 9 to maintain the viability and functionality of the osteocytes 8.

FIG. 2 shows an osteon template comprised of a concentric arrangement 10 of a first plurality of filaments 11, and a second plurality of filaments 12. The first plurality of filaments 11 are vasculogenic filaments composed of a vascular-forming hydrogel, and the second plurality of filaments 12 are osteogenic filaments composed of a bone-forming hydrogel. The first plurality of filaments 11 is arranged alternately with the second plurality of filaments 12. The alternating filaments 11, 12 form a concentric arrangement 10. The concentric arrangement 10 may involve each filament 11, 12 forming an arc of a circle so that the centre of curvature 13 of each filament coincides with the centre of curvature 13 of its radially neighbouring filament(s). The concentric filaments 11, 12 therefore form a sector of a circle about each of the arc's centre of curvature 13. The length of each concentric filament 11, 12 within one sector hence increases as the diameter of the arc increases away from the centre of the sector 13.

In this embodiment the osteon template is shown as including four radial channels 14 emanating from the centre 15 of the concentric arrangement to interrupt the first and second pluralities of vasculogenic and osteogenic filaments 11, 12. This means that the first and second plurality of filaments 11, 12 are arranged concentrically in an incomplete arc of a circle, i.e. divided into four arcuate segments 10a-10d, such that the radial channels 14 devoid of filament are formed where the ends of the arc of each filament 11, 12 do not meet. In this embodiment the channels 14 emanate radially from the centre of curvature 13 of the arcuate segments 10a-10d, which is similarly the centre 15 of the concentric arrangement 10. The channels 14 extends to the outer edge of the concentric arrangement 10.

The radial channel(s) 14 may act as diffusion channel(s) so that the osteon template comprising the concentric arrangement 10 is perfusive. By providing an open space for fluid to travel through, the diffusion channel(s) 14 allow nutrients to perfuse to the interior of the concentric arrangement 10. Perfusion is hence assisted between the edges of the template and the centre the template, as well as increasing the proximity of the interior filaments 11, 12 to nutrients provided by perfusion along each channel 14. Furthermore the channel(s) 14 allow waste products of the biological processes of bone production, resorption and maintenance to be removed from the interior of the concentric arrangement 10. There is a central void 16 generally at the centre 15 of the concentric arrangement 10. The central void 16 further aids with perfusion throughout the osteon template. This central void 16 mimics the osteonic cannel (Haversian cannel) 4 of the osteon structure 3 as seen in FIG. 1c.

The concentric arrangement 10 of the filaments 11, 12 is configured such that each osteogenic filament 12 is bounded by two vasculogenic filaments 11. Put another way, the filaments alternate in the radial direction between the vasculogenic filament 11 and the osteogenic filament 12. In this embodiment, the innermost filament is a vasculogenic filament 11 and the outermost filament is also a vasculogenic filament 11. Such an arrangement increases the perfusiveness of the concentric arrangement 10 as the vascular structures induced to be formed in the region of the vascular-forming filaments will act to supply nutrients to the bone-forming regions.

FIG. 3 shows the constituent parts of the concentric arrangement 10 for the osteon template in greater detail. In this embodiment the concentric arrangement 10 includes eight radial channels 14 emanating from its centre. The vasculogenic filaments 11 are comprised of vascular-forming hydrogel 20, including vessel-forming cells 21, for example endothelial cells such as human umbilical vein endothelial cells (HUVEC). The vascular-forming hydrogel 20 may be a ‘soft’ hydrogel to mimic the stiffness of natural vessels. The vascular-forming hydrogel 20 may therefore be composed of fibrin. The vascular-forming hydrogel 20 may further comprise nanocellulose 22. The nanocellulose acts to guide HUVEC alignment and aids in angiogenesis tube formation.

The osteogenic filaments 12 are comprised of a bone-forming hydrogel 23, including bone-forming cells 24, for example mesenchymal stem cells such as human bone marrow mesenchymal stem cells (hBMSC). The bone-forming hydrogel 23 may further comprise nanohydroxyapatite (nHA) 25 in order to increase the stiffness of the hydrogel as well as increase the bone deposition rates by providing nucleation sites. The nHA also helps to guide the hBMSC osteogenic differentiation. The bone-forming hydrogel 23 may be a ‘stiff’ hydrogel to mimic the stiffness of natural bone. The bone-forming hydrogel 23 may therefore be composed of a fibrin and alginate hydrogel.

It is shown in FIG. 3 that the concentric arrangement 10 is disposed on a base layer 26. The base layer 26 may be composed of a hydrogel such as fibrin (i.e. a cell-free hydrogel). The base layer 26 may be composed of a thermoplastic material such as PCL. The base layer 26 acts as a support to the filaments 11, 12 disposed on top of it. In particular, when the filaments 11, 12 are arranged in multiple segments 10a-10h due to the multiple radial channels 14, the base layer 26 provides a structure for securing the segments in the correct position relative to each other. Where there is only one radial channel 14 the filaments 11, 12 may be rigid enough to maintain their shape without the need of a base layer 26. The base layer 26 can provide structural stability to the concentric arrangement 10 and increase its portability.

The first plurality of filaments 11 (those filaments 11 composed of the vascular-forming hydrogel 20) may be formed in a core-shell configuration 27. The bottom left inset figure of FIG. 3 shows a cross section of a vasculogenic filament 11 taken along the line A-A i.e. perpendicular to the axis of the filament 11. The core-shell structure 27 is configured such that there is a hollow core 27A running along the axis of the filament 11 surrounded by an annular shell 27B of the vascular-forming hydrogel 20 seen in the top right inset. The core-shell structure 27 of the filaments 11 assists with perfusion. This perfusion is shown schematically in FIG. 4, where arrows 28 show the flow of materials through the hollow core 27A.

The second plurality of filaments 12 (those filaments composed of the bone-forming hydrogel 23) are formed in a solid filament configuration.

In order to mimic the configuration of a natural osteon, the diameter of the concentric arrangement 10 may be around 20 mm. The diameter of the vascular-forming filaments 11 may be around 200 um and the diameter of the bone-forming filaments 12 may be around 400 um. The diameter of the bone-forming filament 12 may be twice that of the vascular-forming filament 11 in order to provide the template with increased area of bone-forming region whilst maintaining a preferred distance of 100-200 um between any bone-forming region and a region of vascularisation to ensure sufficient diffusion of oxygen, nutrients, and removal of waste products. The radial channels 14 may have a width of around 2-3 mm. It will be appreciated that FIGS. 2-8 are not shown to scale.

The concentric arrangement 10 can be tailored to a desired size and to achieve the desired perfusiveness. For example, the larger the diameter of the concentric arrangement, the higher the number of filaments will be required to alternate in the radial direction. The concentric arrangement 10 may then include an increased number of radial channels 14 to compensate for the increased filament length of the outer filaments to ensure proximity of the bone-forming filaments 12 to the nutrient supply afforded by the radial channels 14. The configuration of the template may also depend on whether the vasculogenic filaments 11 are constructed using the core-shell structure 27 discussed above. In order to achieve the desired perfusiveness, more radial channels 14 may be incorporated in the concentric arrangement 10 if the core-shell structure 27 is not used, as compared to a configuration of the concentric arrangement 10 in which the core-shell structure 27 is used.

FIG. 5 shows an osteon template 110 formed from multiple filament layers of the concentric arrangement 10 to form a three-dimensional osteon model. One or more filament layers 10 is stacked on top of a first filament layer 10. The filament layers 10 are stacked so as to form an osteon template 110 that is generally cylindrical in shape. In this embodiment the filament layers 10 forming the osteon template 110 have the same configuration as the other filament layers 10 in the template. In other embodiments the filament layers 10 may have different configurations to each other. For example; the number of radial channels may be different, or the same, throughout the filament layers 10, the vasculogenic filaments 11 may be configured to have the core-shell structure 27 in all or only some filament layers 10, the radial position of the vasculogenic filaments 11 and the osteogenic filaments 12 may be the same within the osteon template 110, as illustrated, or may be different between filament layers 10.

In the example shown in FIG. 5, the layers 10 are stacked such that the radial channels 14 are aligned to form a radially and axially extending channel 114 emanating from the centre 115 of the template 110. In other embodiments the filament layers 10 may be stacked such that the radial channels 14 of each filament layer 10 are misaligned. There is a central void 116 extending axially through the middle of the template 110.

The first and second plurality of filaments 11, 12 may be deposited using an additive manufacturing technique in order to form the concentric arrangement 10 and the 3D osteon template 110. In particular, 3D printers are available which are known to be able to print hydrogel structures and which are capable of forming filaments having the core-shell structure 27. Such a 3D printer is the Aspect Biosystems microfluidic based bioprinting technology (RX1). Other 3D printers available in the art may be adapted to be able to form the core-shell structure 27 by adapting the needle through which the hydrogel is deposited.

Using extrusion-based 3D printers with several dispense heads allows for the printing of different materials. By loading the bone-forming hydrogel in a first cartridge of the 3D printer associated with a first dispense head, the vascular-forming hydrogel in a second cartridge of the 3D printer associated with a second dispense head and a structural bioink [such as thermoplastic (e.g. PCL) or a cell-free hydrogel] in a third cartridge of the 3D printer associated with a third dispense head, the printer can dispense layers of the three hydrogels or bioinks according to a CAD design.

The CAD design of the osteon template comprising a base layer is composed of three separate parts. These parts are made with a CAD software (such as Solidworks and SketchUP) and transformed to STL (Standard Tessellation Language) files. The first part is the base layer that can be printed with a structural bioink such as thermoplastic (e.g. PCL) or a cell-free hydrogel. The second part is the osteogenic filaments that will be printed with the bone-forming hydrogel. The third part is the perfuse-able vasculogenic filaments that will be printed with the vascular-forming hydrogel. The printing of the first, second and third parts need not occur in that order, and may occur in any suitable order, combination or alternation as is required to build up the osteon template having the desired structure. By combining these three parts in one structure in the planning software of the 3D printer (software used to import STL files and to slice and create the layers), the different parts can be assigned to different printing dispense heads and printed according to their order in the combined final design.

Using a coaxial dispense head, tubular (hollow) filaments of the vascular-forming hydrogel can be printed. The tubular vasculogenic filaments hence support vascularization and excellent exchange of nutrients and waste products, and can also support implementation as well-defined microchannels into which the bioreactor tubing can be inserted.

The size of the needles used to print the vascular-forming hydrogel and the bone-forming hydrogel is determined according to the need and the application, as such the size of the needles and the size of the resultant vasculogenic filaments and osteogenic filaments may be different when intended for use in in vivo implantation, or for use in in vitro models for drug screening or for use in a bioreactor dynamic culture. For example, for an osteon template intended for use in in vivo implantation a nozzle having an inner diameter of 400 micrometer can be used to print the osteogenic filament and a nozzle having an inner diameter of 200 micrometer can be used to print the vasculogenic filament; and for an osteon template intended for use in a bioreactor system, nozzles having larger inner diameters can be used.

FIG. 6 shows an exemplary bone graft scaffold 612 into which the osteon templates 10, 110 may be disposed. The bone graft scaffold 612 provides a rigid support structure for the osteon template. The bone graft scaffold 612 comprises an inner portion in the form of a centric tube 614 and an outer portion in the form of a contour tube 616. In the illustrated example the centric tube 614 is shown as a triangular tube but the inner portion may be fabricated to have any suitable shape, for example cylindrical. Also, in this example the contour tube 616 is shown as a cylindrical tube but it may have any suitable cross-sectional shape. In particular, the scaffold 612 may be customised to substantially match a target bone and the shape and/or dimensions of the scaffold may be tailored accordingly. Furthermore, the scaffold 612 may include more than one contour tube 616, for example a number of concentric cylinders arranged inside one another. The walls of the centric tube 614 surround a central channel 618 extending through the scaffold 612. An annular cavity 620 is defined between the inner and outer portions 614, 616. The main function of the contour tube 616 is to support material that may be contained and/or grown in the cavity 620, i.e. using the osteon templates 10, 110 of the present invention.

The concentric tube portions 614, 616 are connected by a base plate 622, the base plate 622 is a generally circular disc comprising a central window that is shaped to match the central channel 618. The window comprises an opening in the material of the base plate 622. Fluid can therefore flow longitudinally through the scaffold 612, i.e. through the window and along the central channel 618. The base plate 622 can be made to different diameters, for example to help incorporate the scaffold 612 into a particular device or fit an intended container such as a cell culture plate or bioreactor chamber.

The scaffold 612 may comprise circumferential apertures 626 in both the inner and outer tubes 614, 616. The apertures 626 may be arranged in alternate layers so that a solid layer in the outer tube lies 616 lies in the same circumferential angle as a layer in the inner tube 614 comprising apertures 626. This alternating arrangement of the apertures 626 ensures that there is sufficient radial diffusion available through the scaffold 612 at any given height whilst maintaining required mechanical strength of the scaffold. The apertures 626 can aid in supporting the perfusive nature of any osteon templates 10, 110 disposed within the scaffold by allowing the flow of fluid between the host environment and the template through the scaffold. The outer tube 616 may be fabricated with a double wall thickness as compared to the inner tube 614. This helps to provide the outer tube 616 with increased mechanical strength.

The osteon template 10, 110 described above may be used as a filler material for the cavity 620 to mimic the osteon structure of compact bone. Repeated units of the template 10, 110 may therefore be disposed around the circumference of the cavity 620 in the scaffold 612, while porous filler material may be disposed in the central channel 618 or the central channel 618 may be free from filler material. FIG. 7 shows a schematic plan view of the scaffold 612 in which the concentric arrangement 10, or the 3D template 110 is disposed. The templates 10, 110 are disposed in the annular cavity 620 between the inner and outer portions 614, 616 and sit on the base plate 622. In some embodiments the templates 10, 110 are placed in multiple layers in the annular cavity 620 in order to pack the templates close together, in some embodiments fewer templates are placed in the scaffold.

As is described in WO2018/162764, the scaffold 612 can be customised to match the dimensions of a particular osseous environment in a human or animal target. A device comprising such a customised scaffold 612 may then be used in vivo, for example for a bone graft, or in vitro, for example as a biological model for mimicking and analysing tissue engineering processes or for analysing drug release under controlled circumstances. The custom dimensions of the scaffold 612 may be determined using non-invasive imaging techniques such as CT or MRI for a particular target. Such customisation of the scaffold dimensions can be particularly important for implants as the environment for bone (re)generation is very complicated (including e.g. different cell types, growth factors, nutrition supply and mechanical stimulation) and the more closely the scaffold can mimic the osseous environment the more likely is the implant to be successful in bone (re)generation. Furthermore, the diameter of the osteon templates 10, 110 may be chosen based on a target bone to be grown using the osteon templates 10, 110 supported by the scaffold 612.

As mentioned above, the osteon templates and methods disclosed herein may find use in a variety of applications including, but not limited to:
Implants for tissue engineering, in particular to assist with bone healing;
Biological models for mimicking and analysing tissue engineering processes;
Biological models for analysing drug release under controlled circumstances;
Template-based studies in bioreactors.

Each of these potential uses can take advantage of the unique template configuration.

FIG. 8 shows the osteon template 10 used in a bioreactor assembly. In this embodiment the vasculogenic filaments 11 of the osteon template have the core-shell structure as explained in relation to FIGS. 3 and 4. The bioreactor assists in mimicking the natural cellular environment by forcing perfusion throughout the filaments 11 in a similar way to how fluid is forced to perfuse around natural structures of an animal body. In this way mass transfer and fluid flow is improved around the osteon template 10. A tubular network 700 can be disposed within the osteon template 10. An entrance tube 710 and an exit tube 720 can be disposed within the radial channels 14 of the template 10, and circumferentially extending tubes 730 can be disposed within the core of the vasculogenic filaments 11. The circumferentially extending tubes 730 emanate from the entrance tube 710 and then reconnect to the exit tube 720 at the opposite side of the osteon template 10. An input flow of fluid 740 is introduced to the tubular network 700 to flow along the entrance tube 710 and is directed through the tubular network 700 so that the flow leaves the tubular network 700 via the exit tube 720 as an output flow of fluid 745. The tubular network 700 can be connected to a bioreactor (not shown) to communicate the input and output fluid flows 740, 745.

What is claimed is:

1. An osteon template for tissue engineering, comprising:
a first plurality of vasculogenic filaments comprising a first hydrogel and vessel-forming cells and a second plurality of osteogenic filaments comprising a second hydrogel and bone-forming cells;
wherein the first plurality of vasculogenic filaments is arranged alternately with the second plurality of osteogenic filaments in a concentric arrangement.

2. The osteon template of claim 1, further comprising at least one radial channel emanating from the center of the concentric arrangement to interrupt the first and second pluralities of vasculogenic and osteogenic filaments.

3. The osteon template of claim 2, comprising a number n of radial channels interrupting the first and second pluralities of vasculogenic and osteogenic filaments so as to divide each of the vasculogenic and osteogenic filaments into n arcuate segments.

4. The osteon template of claim 1, further comprising a central void generally at the center of the concentric arrangement.

5. The osteon template of claim 1, wherein one or more of the first plurality of vasculogenic filaments consist of a hollow core surrounded by a shell comprising the first hydrogel and vessel-forming cells.

6. The osteon template of claim 5, wherein one or more bioreactor tubes are arranged to extend through the hollow core of at least some of the vasculogenic filaments.

7. The osteon template of claim 1, wherein one or more of the osteogenic filaments in the concentric arrangement is bounded by a vasculogenic filament disposed on a radially inward side and another vasculogenic filament disposed on a radially outward side.

8. The osteon template of claim 1, wherein the vasculogenic filaments have a first diameter and the osteogenic filaments have a second diameter that is greater than the first diameter.

9. The osteon template of claim 8, wherein the first diameter is in the range of 50-200 microns and the second diameter is in the range of 200-400 microns.

10. The osteon template of claim 1, wherein the vasculogenic filaments comprise the first hydrogel having a first stiffness and the osteogenic filaments comprise the second hydrogel having a second stiffness that is greater than the first stiffness.

11. The osteon template of claim 1, further comprising a base layer and the concentric arrangement being disposed on the base layer.

12. The osteon template of claim 1, wherein the concentric arrangement forms a first filament layer, the osteon template comprising one or more further filament layers formed from such concentric arrangements and stacked on the first filament layer to form a three-dimensional osteon template comprising a plurality of the filament layers.

13. The osteon template of claim 12, wherein the three-dimensional osteon template is generally cylindrical.

14. The osteon template of claim 12, wherein one or more radial channels are formed in each filament layer and the one or more radial channels are aligned in each of the plurality of filament layers to define an axially and radially extending channel in the three-dimensional osteon template.

15. The osteon template of claim 12, wherein each filament layer comprises a central void and the filament layers are stacked such that the central voids define an axial channel at the centre of the three-dimensional osteon template.

16. The osteon template of any of claim 12, wherein an additional support layer is arranged amongst the plurality of the filament layers.

17. An osteogenic model, comprising a three-dimensional scaffold and a plurality of the osteon templates of claim 12 arranged to be supported by the scaffold in a generally concentric array.

18. The osteogenic model of claim 17, wherein the three-dimensional scaffold comprises a first set of one or more walls and a second set of one or more walls arranged to substantially surround the first set of one or more walls with a spacing between the first and second sets of one or more walls defining a cavity between the walls, and wherein the concentric array is arranged in the cavity between the walls.

19. A method of making the osteon template of claim 1, using additive manufacturing to deposit at least the first plurality of vasculogenic filaments and the second plurality of osteogenic filaments.

20. A method of manufacturing an osteon template, the method comprising:
using an additive manufacturing process to deposit a concentric arrangement comprising a first plurality of vasculogenic filaments and a second plurality of osteogenic filaments, the first plurality of vasculogenic filaments being arranged alternate with the second plurality of osteogenic filaments in the concentric arrangement;
wherein the first plurality of vasculogenic filaments comprises a first hydrogel and vessel-forming cells and the second plurality of osteogenic filaments comprises a second hydrogel and bone-forming cells.

21. The method of claim 20, further comprising:
controlling the additive manufacturing process to form at least one radial channel emanating from the centre of the concentric arrangement to interrupt the first and second pluralities of vasculogenic and osteogenic filaments.

22. The method of claim 20, further comprising:
controlling the additive manufacturing process to form one or more of the first plurality of vasculogenic filaments as a core-shell structure consisting of a hollow core surrounded by a shell comprising the first hydrogel and vessel-forming cells.

23. The method of claim 22, further comprising:
controlling the additive manufacturing process to form a number n of radial channels, wherein n is determined based on how many of the vasculogenic filaments are formed with the core-shell structure.

24. The method of claim 20, further comprising:
determining a diameter for the osteon template based on a target bone to be grown using the osteon template; and
controlling the additive manufacturing process such that the concentric arrangement is formed with said diameter.

25. The method of claim 24, further comprising:
controlling the additive manufacturing process to form a number n of radial channels, wherein n is determined based on the diameter of the concentric arrangement.
